(19) United States
(12) Patent Application Publication
(10) Pub. No.: US 2022/0205986 A1
(43) Pub. Date: Jun. 30, 2022

(54) METHOD OF CONVERSION AND SYNTHESIS OF MATERIALS VIA LABELED BIOSTRUCTURES WITH INORGANIC MATERIALS CONJUGATED ANTIBODY

(71) Applicant: Korea Advanced Institute of Science and Technology, Daejeon (KR)

(72) Inventors: Jae Byum CHANG, Daejeon (KR); Dae Hyeon SONG, Daejeon (KR); Chang Woo SONG, Daejeon (KR)

(21) Appl. No.: 17/560,654

(22) Filed: Dec. 23, 2021

(30) Foreign Application Priority Data
Dec. 28, 2020 (KR) 10-2020-0184113
Aug. 19, 2021 (KR) 10-2021-0109598
Dec. 1, 2021 (KR) 10-2021-0169688

(51) Int. Cl. G01N 33/536; G01N 33/533; G01N 33/551; G01N 1/30

(52) U.S. Cl. CPC G01N 33/536; G01N 1/30; G01N 33/551; G01N 33/533

(57) ABSTRACT

Various embodiments provide a method of conversion and synthesis of materials via biostructures labeled with an antibody which an inorganic particle has been conjugated. According to various embodiments, a structure corresponding to a biostructure is generated by labeling an antibody which an inorganic particle has been conjugated in a biostructure and growing the inorganic particle with respect to the biostructure.

### CROSS-REFERENCES TO RELATED APPLICATIONS

This application is based on and claims priority under 35 U.S.C. 119 to Korean Patent Application Nos. 10-2020-0184113, filed on Dec. 28, 2020, 10-2021-0109598, filed on Aug. 19, 2021, and 10-2021-0169688, filed on Dec. 1, 2021 in the Korean intellectual property office, the disclosures of each of which are herein incorporated by reference in their entireties.

### TECHNICAL FIELD

Various embodiments relate to a method of conversion and synthesis of inorganic structural materials via biostructures, and more particularly, to a method of. conversion and synthesis of inorganic structural materials from complex biostructures through labeling using an antibody, which a gold nano particle is conjugated, and particle growth.

### BACKGROUND OF THE DISCLOSURE

Biostructures have highly-organized hierarchical structures, which have excellent physical properties, from a nano scale to a macro scale. If metal or inorganic materials are synthesized by using a biostructure as a template, it is expected that the synthesis of structural materials which have extraordinary properties is possible. However, according to the existing bio-templating research, only a simple structure can be implemented by using a virus, a microorganism, etc. as a template, but various internal hierarchical structures of cells and tissues cannot be used as a template.

### SUMMARY

This summary is provided to introduce a selection of concepts in a simplified form that is further described below in the Detailed Description. This summary is not intended to identify key features of the claimed subject matter, nor is it intended to be used as an aid in determining the scope of the claimed subject matter.

Various embodiments provide a method of conversion and synthesis of inorganic structural materials via biostructures.

Various embodiments provide a method of conversion and synthesis of inorganic structural materials via biostructures labeled with an antibody to which an inorganic particle has been conjugated.

According to various embodiments, a method of conversion and synthesis of inorganic structural materials via biostructures may include steps of labeling biomolecules in a biostructure through an antibody which an inorganic particle has been conjugated, and generating inorganic structural materials corresponding to the biostructure by growing an inorganic particle.

According to various embodiments, structures having functional materials can be manufactured using a complex biostructure as a template in a way to stain a biostructure within a biomaterial by using an antibody and grow an inorganic particle from the antibody. In this case, through the selection of the inorganic particle, a specific characteristic can be further increased or a combination of several characteristics can be implemented with respect to the structure. Also, through immunostaining using an antibody pair, the structure can be manufactured in a form modified from the biostructure. Furthermore, the inorganic particle can be grown in various forms, and can be synthesized into multi-material particles by applying another material. That is, the inorganic structure can be manufactured from a biostructure according to the desired application purpose.

### DESCRIPTION OF THE DRAWINGS

The foregoing aspects and many of the attendant advantages of this disclosure will become more readily appreciated as the same becomes better understood by reference to the following detailed description, when taken in conjunction with the accompanying drawings, wherein:

FIG. 1 is a flowchart illustrating a method of conversion and synthesis of materials via biostructures according to various embodiments.

FIG. 2A is a flowchart illustrating a method of conversion and synthesis of materials via biostructures according to various embodiments.

FIG. 2B is an exemplary diagram for exemplarily describing a method of conversion and synthesis of materials via biostructures according to various embodiments.

FIGS. 3A and 3B are diagrams for exemplarily describing a method of conversion and synthesis of materials via biostructures according to some embodiments.

FIGS. 4A, 4B, and 4C are diagrams for exemplarily describing a method of conversion and synthesis of materials via biostructures according to some embodiments.

FIG. 5 is a diagram for exemplarily describing methods of conversion and synthesis of materials via biostructures according to some embodiments.

FIGS. 6 to 20 are diagrams for describing experimental implementations and demonstration results of various embodiments.

### DETAILED DESCRIPTION

While illustrative embodiments have been illustrated and described, it will be appreciated that various changes can be made therein without departing from the spirit and scope of the disclosure.

Hereinafter, various embodiments of this document are described with reference to the accompanying drawings.

Various embodiments provide a method of conversion and synthesis of inorganic structural materials via inside or outside biostructures of biological specimens, and provide method of conversion and synthesis of an inorganic structural material via a complex biostructure through labeling using an antibody which gold nano particles are conjugated and particle growth.

According to various embodiments, a biological specimen, that is, a target, may include prokaryotes such as viruses and bacteria, unicellular eukaryotes such as yeast, protoctists such as diatoms, insects, animal tissues, a plant, and human body tissues. The biological specimen may include cultured cells, cultured tissues, organoids, etc. which are artificially cultured on a plane or scaffold. Furthermore, the biological specimen may include 3D-printed cells or tissues, stacks of tissues, or an organ-on-a-chip obtained by culturing cells or tissues on a chip. Also, the biological specimen may include such biological specimens on which gene-editing or processing has been performed.

According to various embodiments, a biostructure labeled with an antibody in biological specimens may be composed of lipids, nucleic acids, inorganic materials, etc., including proteins.

According to various embodiments, since immunostaining is used, various biostructures can be labeled and also converted into inorganic materials by simultaneously using several antibodies. Various inorganic particles may be first conjugated with antibodies, and the biostructures may be converted into structural materials composed of various inorganic materials by specifically labeling several types of proteins with the antibodies.

According to various embodiments, inorganic particles can be synthesized in the form of a biostructure modified from an original structure through various types of antibodies and immunostaining strategies.

According to various embodiments, an antibody may include a common antibody, an antibody fragment, such as F(ab)₂, F(ab′)₂, Fab′, Fab, Fv, scFv, or VhH, and a minibody, a nanobody, affibody, diabody, and may include a nucleic acid-based aptamer.

According to various embodiments, an antibody may be substituted with any molecules capable of selectively labeling only a specific structure, for example, a nucleic acid sequence complementarily binding to a specific base sequence, a hydrophobic molecule selectively labeling lipids, etc.

According to various embodiments, instead of using inorganic particle-conjugated antibodies, a target molecule is expressed within cells through gene-editing and the target molecule is labeled with inorganic particle-conjugated another molecule that specifically binds to the target molecule.

According to various embodiments, a biostructure can be converted and synthesized into multi-material particles, such as core-shell particles, not a single particle, through various inorganic particle synthesis in an aqueous solution. Furthermore, a biostructure can be converted and synthesized into particles having various shapes through various particle synthesis strategies.

Various embodiments have 1) an advantage in that complex and elaborate structures of biostructures difficult to be manufactured through existing three-dimensional structure manufacturing technologies can be manufactured and 2) a distinction in that various materials can be introduced into a structure. Furthermore, by overcoming the limits of the current bio-templating methods that imitate only a surface of the existing biostructure, 3) a specific biostructure present within a cell or a tissue can be converted and synthesized into another material. Accordingly, various embodiments present a paradigm of a new bio-templating method and have the capability that it can be used in several fields.

Various embodiments may be used in a wide manner by being combined with 1) immunostaining methods, 2) a particle growth techniques, or 3) genetic/biotechnology. For example, various embodiments may include 1) a method of more finely synthesizing a structural material of another material via a biostructure by labeling the biostructure with an antibody having a small form such as a nano body, 2) a method of synthesizing materials having various forms such as a gold by-pyramid structure, 3) a method of generating a complex structural materials of another material via biostructure not present in the natural world through tissue engineering technologies, such as gene-editing technologies, three-dimensional cell cultures, or printing technologies, etc. Accordingly, according to various embodiments, efficient and various structural materials corresponding with an application can be generated and used in a wide market.

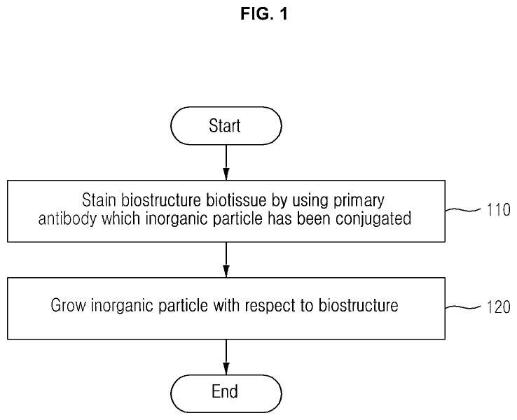

FIG. 1 is a flowchart illustrating a method of conversion and synthesis of materials via biostructures according to various embodiments.

Referring to FIG. 1, in step 110, a biostructure inside or outside a biological specimen is stained by using a primary antibody which an inorganic particle has been conjugated. For example, after a target protein forming a structure to be imitated within a biological specimen is selected, a primary antibody is selectively combined with an antigen of the target protein through antigen/antibody reactions. Accordingly, as a biostructure, such as the target protein, is labeled with the primary antibody, the inorganic particle is disposed in the biostructure. In this case, the inorganic particle includes at least one of colloidal particles, nano particles, metal particles, non-conductive particles, semiconductor particles, quantum dots, or carbon particles. For example, the inorganic particle is a gold nano particle.

Next, in step 130, the inorganic particle is grown with respect to the biostructure. For example, the size of the inorganic particle is increased or the inorganic particle generates material growth for desired material particles. Accordingly, as the inorganic particle is grown, an inorganic structural material corresponding to the biostructure is generated. That is, the structure having the desired form in which the complexity, structural characteristic, etc. of the biostructure are maintained is generated.

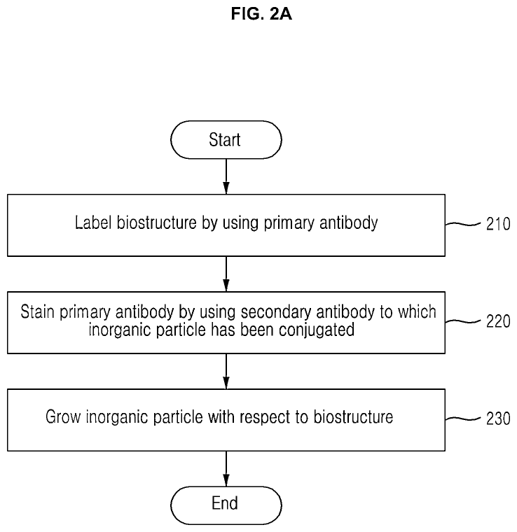

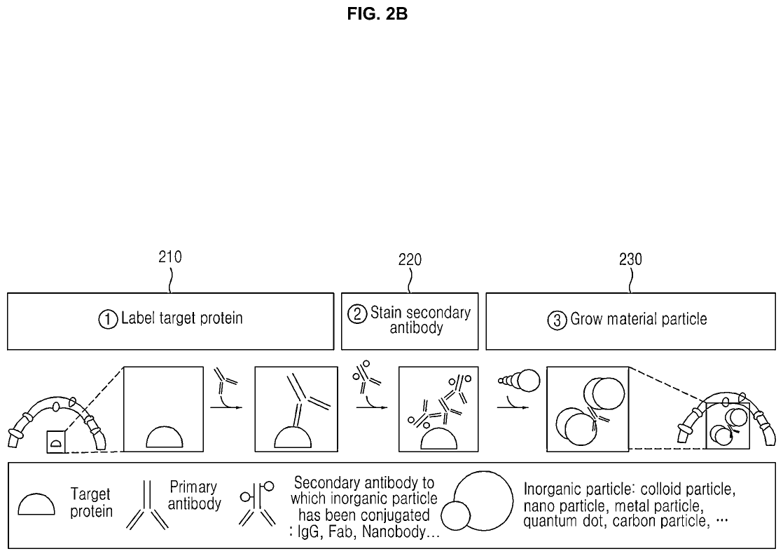

FIG. 2A is a flowchart illustrating a method of conversion and synthesis of materials via biostructures according to various embodiments. FIG. 2B is an exemplary diagram for exemplarily describing a method of conversion and synthesis of materials via biostructures according to various embodiments.

Referring to FIG. 2A, in step 210, a biostructure inside or outside a biological specimen is labeled with a primary antibody. For example, as illustrated in FIG. 2B, after a target protein forming a structure to be imitated within a biological specimen is selected, a primary antibody is selectively combined with an antigen of the target protein through antigen/antibody reactions. Accordingly, a biostructure, such as the target protein, are labeled with the primary antibody.

Next, in step 220, the biostructure is stained by using a secondary antibody which an inorganic particle has been conjugated. In this case, the secondary antibody is combined with the primary antibody attached to the biostructure. For example, as illustrated in FIG. 2B, the target protein labeled with the primary antibody is stained by using the secondary antibody which the inorganic particle has been conjugated. Accordingly, the inorganic particle is disposed in the biostructure such as the target protein. In this case, the inorganic particle includes at least one of colloidal particles, nano particles, metal particles, non-conductive particles, semiconductor particles, quantum dots, or carbon particles. For example, the inorganic particle is gold nano particles.

Next, in step 230, the inorganic particle is grown with respect to the biostructure. For example, as illustrated in FIG. 2B, the size of the inorganic particle is increased or the inorganic particle generates material growth for desired material particles. Accordingly, as the inorganic particle is grown, an inorganic structural material corresponding to the biostructure is generated. That is, the structure having a desired form in which the complexity, structural characteristic, etc. of the biostructure are maintained is generated.

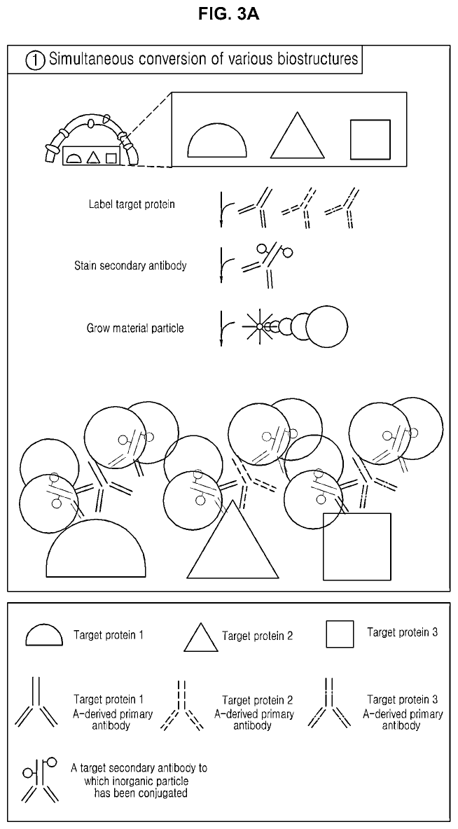

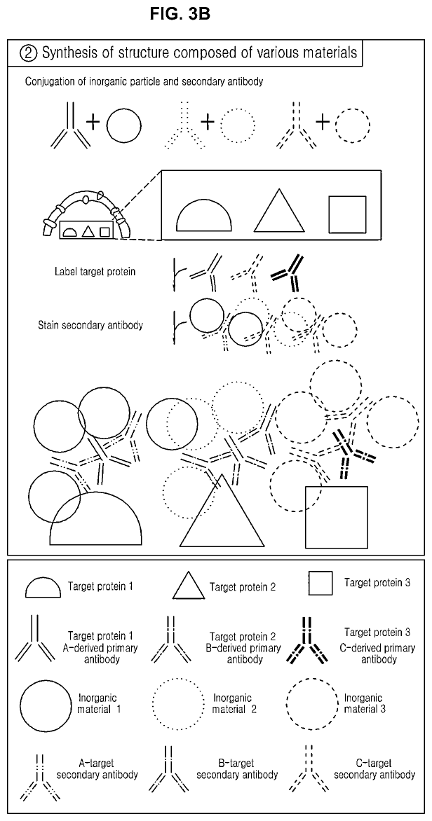

FIGS. 3A and 3B are diagrams for exemplarily describing a method of conversion and synthesis of materials via biostructures according to some embodiments.

Referring to FIGS. 3A and 3B, in some embodiments, the aforementioned method of conversion and synthesis of materials via biostructures is implemented with respect to several biostructures within the same biological specimen. FIG. 3A illustrates a method of conversion and synthesis of materials via biostructures according to a first embodiment, and FIG. 3B illustrates a method of conversion and synthesis of materials via biostructures according to a second embodiment.

According to the first embodiment, different biostructures within the same biological specimen are simultaneously converted and synthesized into the same material. For example, as illustrated in FIG. 3A, after different biostructures are labeled with different primary antibodies, the primary antibodies are stained by using a secondary antibody which the same inorganic particle has been conjugated. Accordingly, as an inorganic particle is simultaneously grown in different biostructures, a structure having the same material is generated with respect to different biostructures. In such a case, a structure is manufactured from different biostructures at a time. A characteristic of a material can be used with further improved efficiency through the structures.

According to the second embodiment, different biostructures within the same biological specimen are converted and synthesized into different materials. For example, as illustrated in FIG. 3B, after different biostructures are labeled with different primary antibodies, the primary antibodies are stained by using different secondary antibodies which different inorganic particles have been conjugated, respectively. Accordingly, as the different biostructures are individually converted into different inorganic materials, structures having different materials are generated with respect to the different biostructures. In such a case, the different materials are simultaneously disposed in the different biostructures. A combination of characteristics of the different materials used will be used through the structures.

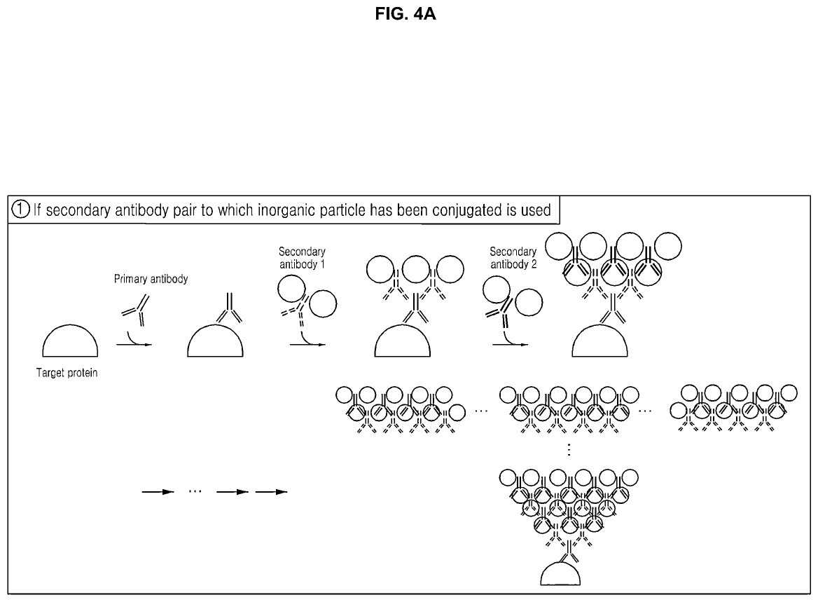

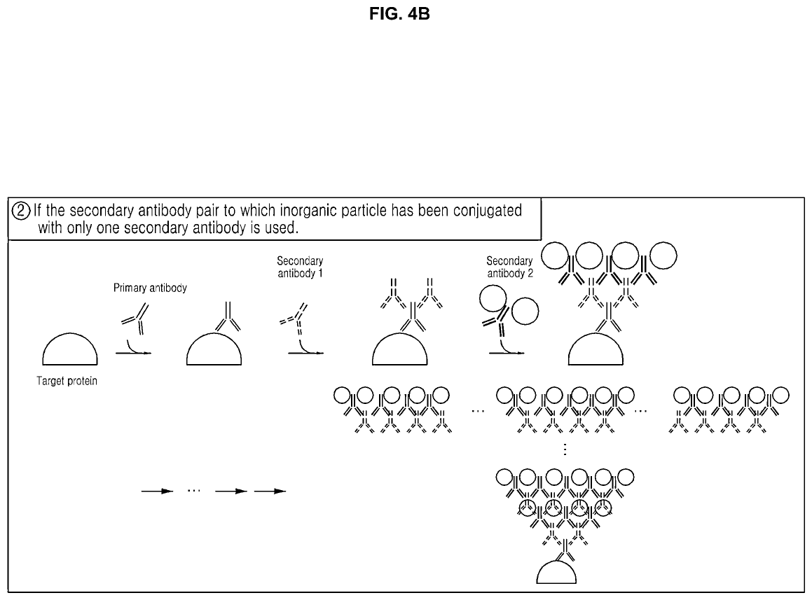

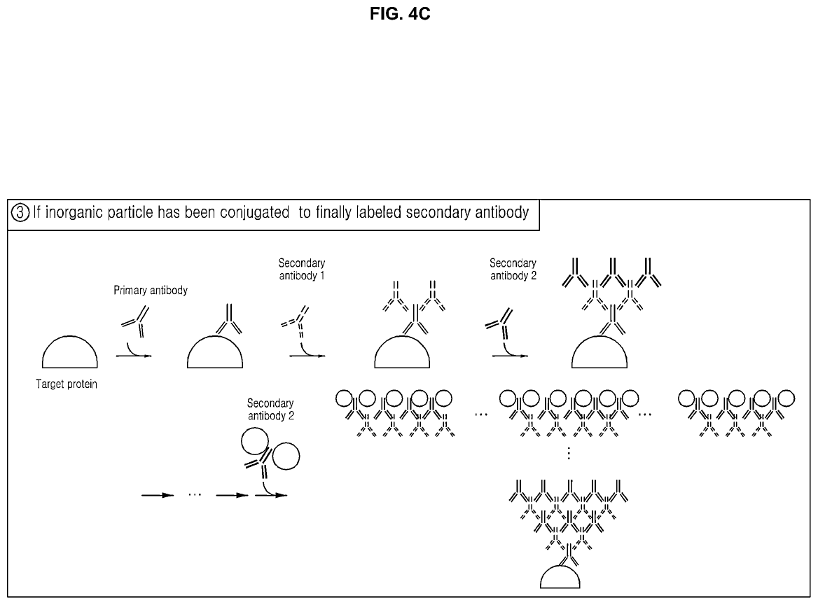

FIGS. 4A, 4B, and 4C are diagrams for exemplarily describing a method of conversion and synthesis of materials via biostructures according to some embodiments.

Referring to FIGS. 4A, 4B, and 4C, in some embodiments, the aforementioned method of conversion and synthesis of materials via biostructures is implemented even in a form modified from the biostructure through immunostaining using an antibody pair. FIG. 4A illustrates a method of conversion and synthesis of materials via biostructures according to a third embodiment, FIG. 4B illustrates a method of conversion and synthesis of materials via biostructures according to a fourth embodiment, and FIG. 4C illustrates a method of conversion and synthesis of materials via biostructures according to a fifth embodiment.

According to some embodiments, after biostructures are labeled with a primary antibody, the primary antibody is repeatedly stained by using a secondary antibody pair. That is, after the primary antibody is stained by using the secondary antibody, the stained secondary antibody is additionally stained by using another secondary antibody. In this case, the primary antibody is stained by using secondary antibody pairs as a plurality of layers by repeating that the stained secondary antibody is additionally stained by using another secondary antibody more than once.

The secondary antibody pair includes a first secondary antibody and a second secondary antibody. For example, as illustrated in FIGS. 4A, 4B, and 4C, after biostructures are labeled with a primary antibody, the primary antibody is stained using a secondary antibody pair. Specifically, the primary antibody is stained using a first secondary antibody, and the first secondary antibody is stained using a second secondary antibody. Thereafter, the stained secondary antibody pair is additionally stained again by using the secondary antibody pair. Specifically, the stained secondary antibody pair is stained using the first secondary antibody, and the first secondary antibody is stained using the second secondary antibody. In this case, the primary antibody is stained using the secondary antibody pairs as a plurality of layers by repeating that the stained secondary antibody is additionally stained using another secondary antibody more than once.

According to the third embodiment, an inorganic particle has been conjugated with both a first secondary antibody and second secondary antibody of a secondary antibody pair. For example, as illustrated in FIG. 4A, a primary antibody is stained using secondary antibody pairs as a plurality of layers. Inorganic particles have been conjugated with both first secondary antibodies and second secondary antibodies of all the layers. Accordingly, as inorganic particle is grown in biostructures, a structure is generated. In such a case, the structure thicker than the original thickness of the biostructure is manufactured.

According to the fourth embodiment, an inorganic particle is not conjugated with a first secondary antibody, and is conjugated with only a second secondary antibody in a secondary antibody pair. For example, as illustrated in FIG. 4B, a primary antibody is stained using secondary antibody pairs as a plurality of layers, and an inorganic particle is attached to only second secondary antibodies of all the layers. Accordingly, the inorganic particle is grown in a biostructure, and a structure is generated. In such a case, the structure thicker than an original thickness of a biostructure is manufactured, and the structure is manufactured as an inorganic material stack structure.

According to the fifth embodiment, an inorganic particle has been conjugated with only a secondary antibody of the last layer pair. That is, the inorganic particle has not been conjugated with the secondary antibody not the last layer. For example, as illustrated in FIG. 4C, a primary antibody is stained using secondary antibody pairs as a plurality of layers, and an inorganic particle is conjugated with only a secondary antibody of the last layer. Accordingly, the inorganic particle is grown in a biostructure, and a structure is generated. In such a case, the structure thicker than an original thickness of a biostructure is manufactured, and the structure is manufactured as a hollow type structure.

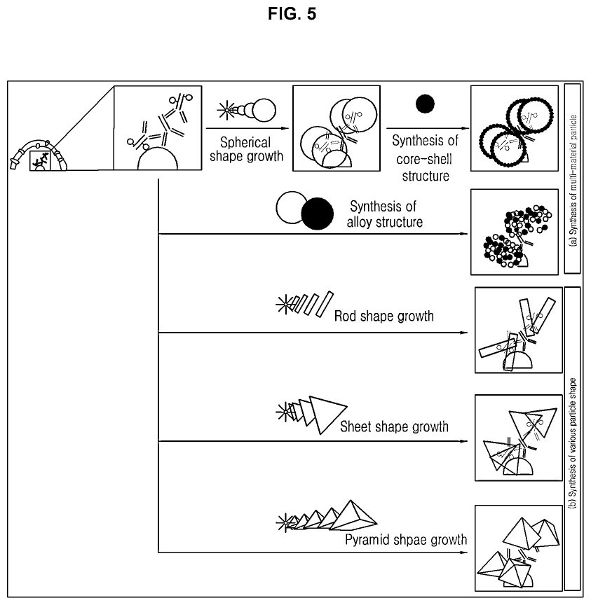

FIG. 5 is a diagram for exemplarily describing a method of conversion and synthesis of materials via biostructures according to some embodiments.

Referring to FIG. 5, in some embodiments, as an inorganic particle is grown based on various growth strategies, the aforementioned method of conversion and synthesis of materials via biostructures is implemented. FIG. 5 illustrates methods of conversion and synthesis of materials via biostructures according to sixth and seventh embodiments.

According to the sixth embodiment, a multi-material particle is synthesized from an inorganic particle. Specifically, as illustrated in FIG. 5(a), at least one different material is applied to a biostructure along with an inorganic particle, and a multi-material particle is synthesized. Accordingly, a structure composed of multi-material particle is generated. For example, after an inorganic particle is grown with respect to a biostructure, at least one different material is applied to the outside of the grown inorganic particle. Accordingly, the multi-material particle is synthesized as a core-shell structure. For another example, various types of materials are simultaneously used as inorganic particles with respect to biostructures. Accordingly, a multi-material particle is synthesized as an alloy structure.

According to the seventh embodiment, an inorganic particle is grown in various forms. Specifically, an inorganic particle may be grown to have a form of a spherical shape, such as that illustrated in FIG. 5(a), or may be grown to have a form of a rod shape, a sheet shape, or a pyramid shape, such as that illustrated in FIG. 5(b). According to the sixth and seventh embodiments, a structural characteristic of a biostructure can be used through a structure, and physical and chemical characteristics can be adjusted through a grown inorganic particle in the structure.

Differentiated characteristics of a method according to various embodiments are listed as follows.

The first characteristic is to enable the synthesis of a high-speed/precision three-dimensional structural material via a biostructure. In general, lithography or printing technologies are used in three-dimensional structure manufacturing. Such technologies have a time-consuming process and require a high cost in order to synthesize a structure having high resolution. Alternatively, if such technologies have a high manufacturing speed, there is a problem in that resolution is low. Accordingly, there is a difficulty in that a scale or complexity of a biostructure is incorporated through the existing three-dimensional structure manufacturing technology. However, a method according to various embodiments has advantages in that a high manufacturing speed because immunostaining and particle growth on an aqueous solution are used and a structure can be synthesized without being limited to the number of biostructures, a scale or complexity because an inorganic particle is generated in a biostructure through antibodies.

The second characteristic is to enable the imitation of a biostructure within a cell/tissue. The existing bio-templating methods do not incorporate varieties of biostructures because material synthesis is performed on an external structure of the biostructure. However, in a method according to various embodiments, since an antibody is used, a biostructure present within a cell or tissue, including an external structure of the biostructure, can be imitated, and characteristics of the biostructures can be differently used.

The third characteristic is to enable the synthesis of multiple structures and multiple materials within a sample. Cells or tissues form various different biostructures as several types of protein. The existing method of synthesizing a three-dimensional structure has increased complexity if multiple structures within the same sample are generated. Furthermore, there is a great difficulty if a structure is generated using other materials according to structures. However, in a method according to various embodiments, synthesis is possible regardless of the number of structures within the same sample by using antibodies capable of labeling different proteins within a cell or a tissue. Furthermore, multiple materials can be synthesized for each structure within the same sample by differentiating the type or form of a material in an antibody that labels each structure.

The fourth characteristic is to facilitate the modification of a structure. The existing three-dimensional manufacturing technology has difficulty in the modification of the first designed structure because a mask is manufactured or manufactured based on a standardized frame. Furthermore, the existing bio-templating methods have a disadvantage in that only the manufacturing of an original form of a biostructure is possible. However, in a method according to various embodiments, synthesis is possible in the form of an original structure by using immunostaining. Furthermore, a scale of an original structure can be easily adjusted or a stacked structure and a hollow structure can be easily manufactured through the repeated labeling of a secondary antibody.

The fifth characteristic is to enable the application of various genetic/bioengineering technologies. With the development of a gene editing technology so far or a genetic/bioengineering technology, such as organoid manufacturing through three-dimensional cell culturing or printing, technologies in which an experimenter generates biological specimens having the desired form while maintaining the complexity and structural characteristic of a biostructure are developed. If a method according to various embodiments is used for such technologies, various structural materials can be manufactured by converting and synthesizing via biostructures not present in the natural world.

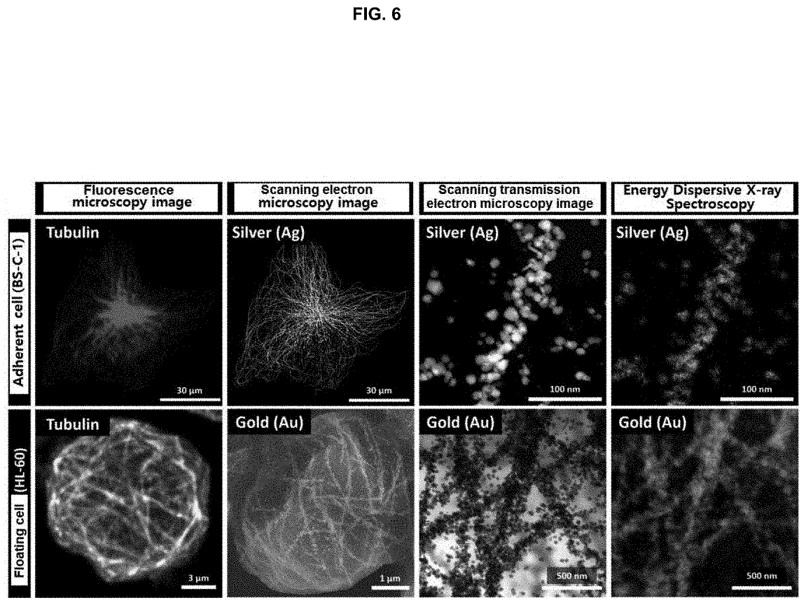

FIGS. 6 to 20 are diagrams for describing experimental implementations and demonstration results of various embodiments. As will be described later, various embodiments have been experimentally implemented and demonstrated, an example used as a catalyst and a surface-enhanced Raman scattering substrate has been illustrated. It was confirmed that the present disclosure is a competitive technology which may be used in various fields.

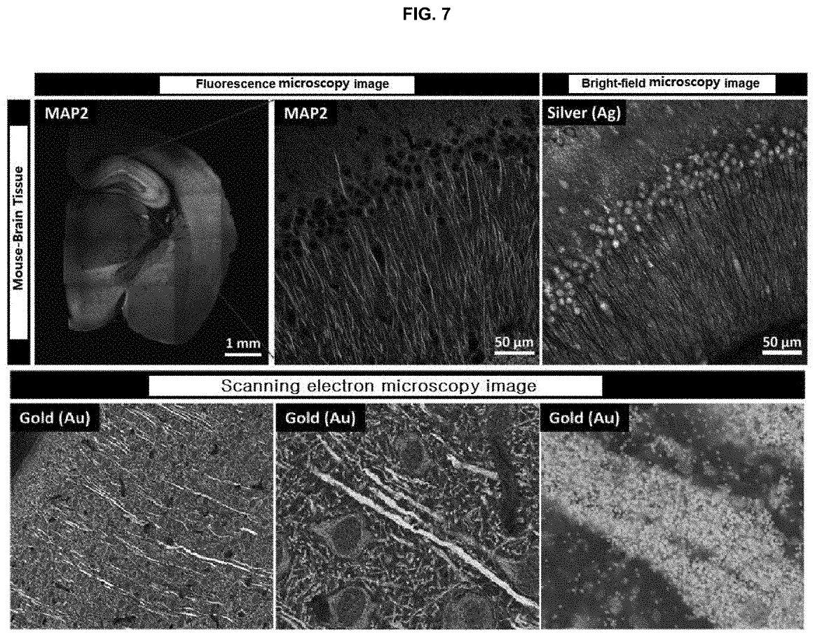

In the first experiment, the conversion and synthesis of materials via biostructures were confirmed by applying a method according to various embodiments. Specifically, a gold, silver structure was synthesized by labeling microtubules within cells. Furthermore, a gold, silver structure was synthesized by labeling microtubule associated protein (MAP2) in a mouse brain tissue. Accordingly, it was confirmed that the conversion and synthesis of a material via a biostructure labeled with an antibody which an inorganic particle has been conjugated operated. In this case, as illustrated in FIG. 6, it was confirmed that a target protein was accurately labeled by using an inorganic particle and an antibody which a fluorescent molecule was conjugated through fluorescence microscopy. Thereafter, it was confirmed that an inorganic particle was synthesized depending on a structure through scanning electron microscopy and scanning transmission electron microscopy and that growth occurred as a desired inorganic particle through energy dispersive X-ray spectroscopy analysis (EDX). Furthermore, as illustrated in FIG. 7, it was confirmed that an inorganic particle was synthesized depending on the structure of target protein in a cell and further even in a mouse brain tissue through images of fluorescence microscopy, bright-field microscopy and scanning electron microscopy.

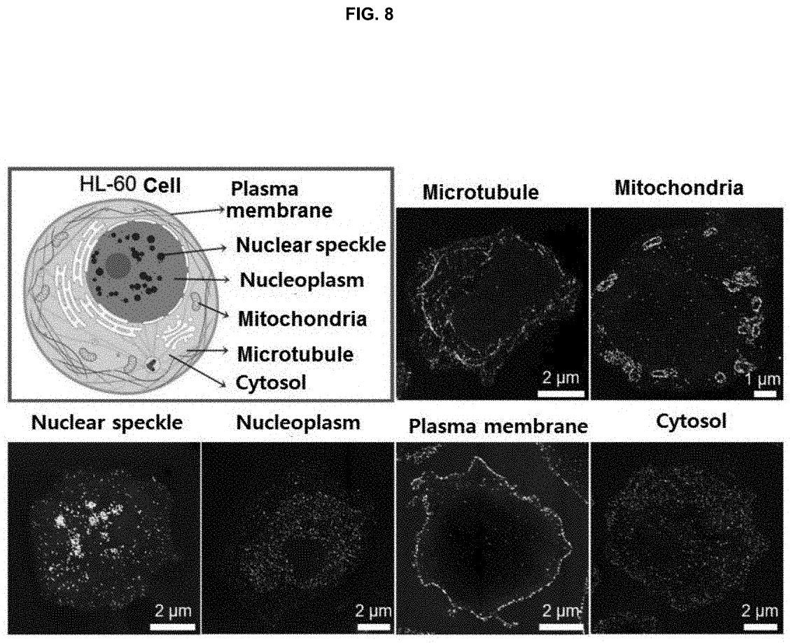

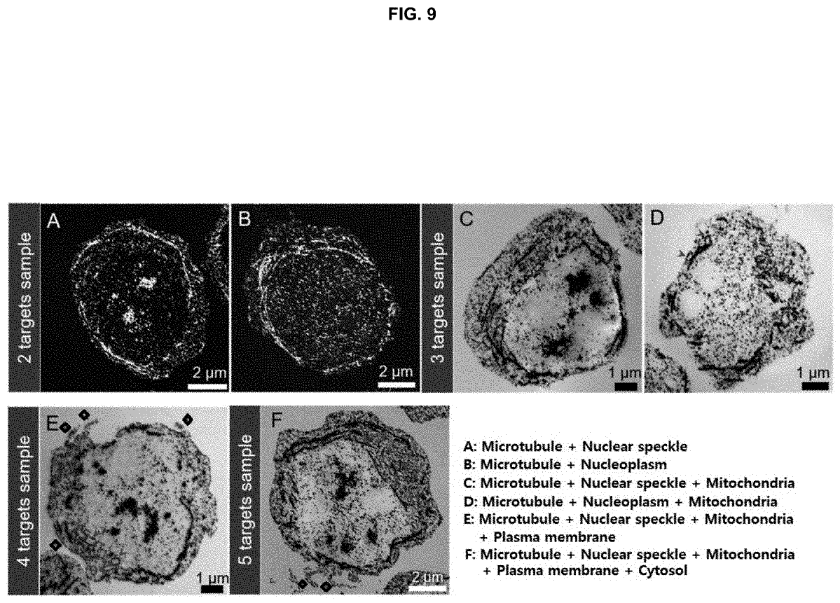

In the second experiment, it was confirmed that materials are simultaneously converted and synthesized from various biostructures within cells. Specifically, as illustrated in FIG. 8, by using different antibodies capable of labeling different biostructures within a cell, five different types of biostructures (mitochondria, a nuclear speckle, a nucleoplasm, a plasma membrane, and cytosol) including a microtubule were labeled, and an inorganic particle was synthesized depending on each structure. Furthermore, as illustrated in FIG. 9, biostructures were labeled with several combinations and inorganic material structures were simultaneously synthesized by performing gold particle growth, through a method according to various embodiments.

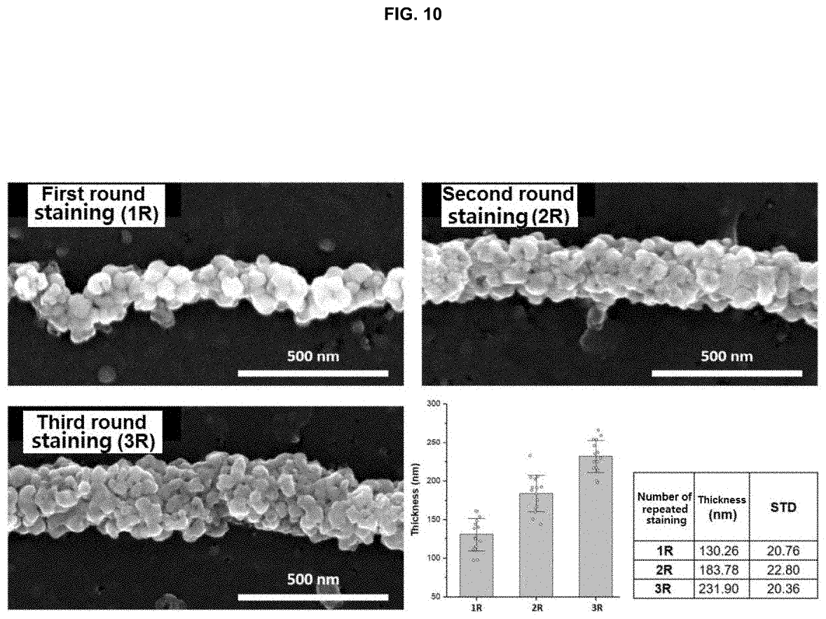

In the third experiment, the synthesis of materials of biostructures through repetitive antibody staining was confirmed. Specifically, as illustrated in FIG. 10, while a repetitive labeling process of a secondary antibody pair which an inorganic particle was conjugated not a single labeled antibody was repeated, the inorganic particle was synthesized in the form of a structure modified from an original biostructure. It was confirmed that since a microtubule within a cell formed a linear structure, the linear structure was maintained even after particle growth occurred and the thickness of the linear structure was increased through the labeling process of the repetitive antibody pair.

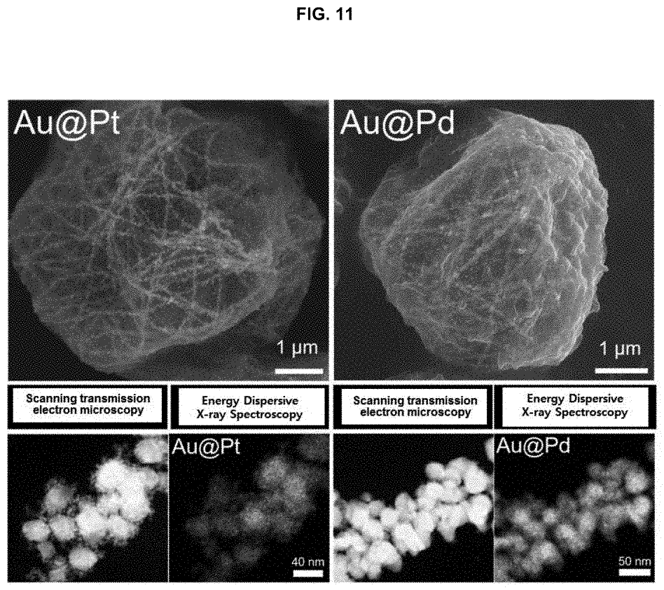

In the fourth experiment, the synthesis of materials via biostructures through core-shell particle synthesis was confirmed. Specifically, as illustrated in FIG. 11, an inorganic material structure was synthesized by using a biostructure as a core-shell particle through a multi-material particle synthesis strategy not the synthesis of a single material particle. A gold particle was grown along a microtubule within a floating cell, and gold@platinum, gold@palladium core-shell particles were synthesized through platinum and palladium synthesis processes, respectively. This shows that various particle growth strategies including multi-material particle growth not only single material particle growth are possible through a method according to various embodiments.

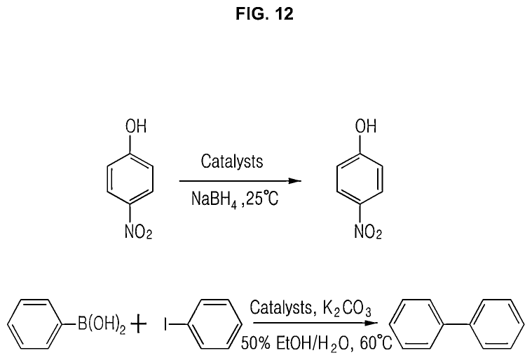

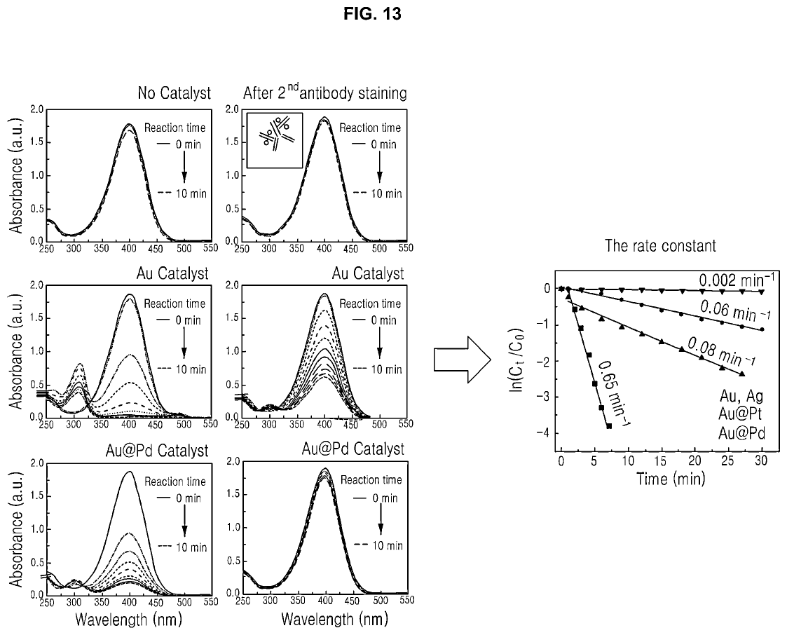

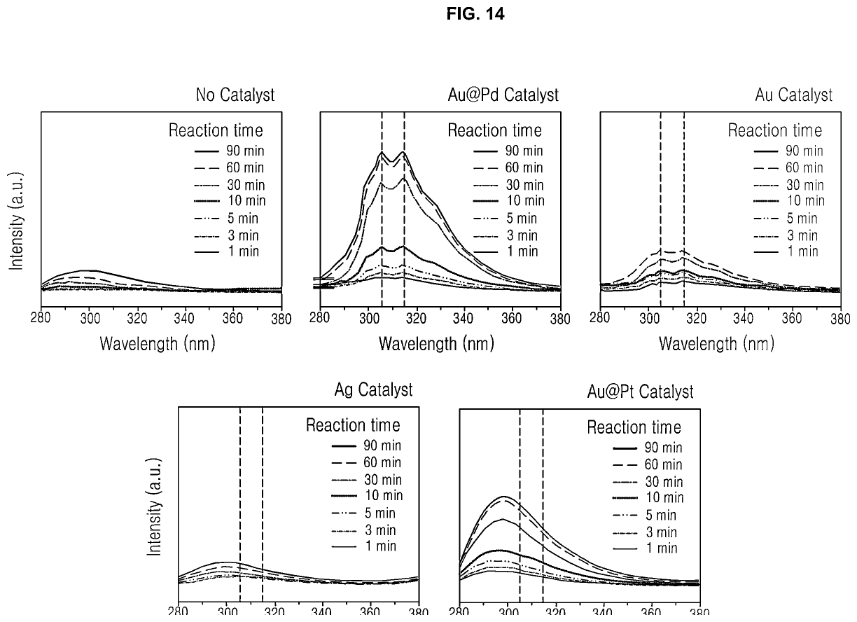

In the fifth experiment, an application as a catalyst for liquid-phase reaction through a method according to various embodiments was confirmed. A floating cell in which metal particles were synthesized through a method according to various embodiments was used as catalysts for various liquid-phase reactions. The first reaction performed was a 4-nitrophenol reduction reaction by using sodium borohydride (NaBH₄) as a reducing agent, such as that illustrated in FIG. 12. The second reaction is a suzuki-miyaura reaction in which coupling between iodobenzene and phenylboronic acid occurs, such as that illustrated in FIG. 12. A cell including metal particles synthesized through a method according to various embodiments well operated even as catalysts of the two liquid-phase reactions. Specifically, as illustrated in FIG. 13, gold particle-grown cells have the highest catalyst activity in the 4-nitrophenol reduction reaction. As illustrated in FIG. 14, the catalyst activity of the suzuki-miyaura reaction was the best in the gold@palladium core-shell particle-grown cells. Such results show that a cell synthesized through a method according to various embodiments can be used as catalysts of various liquefied reactions.

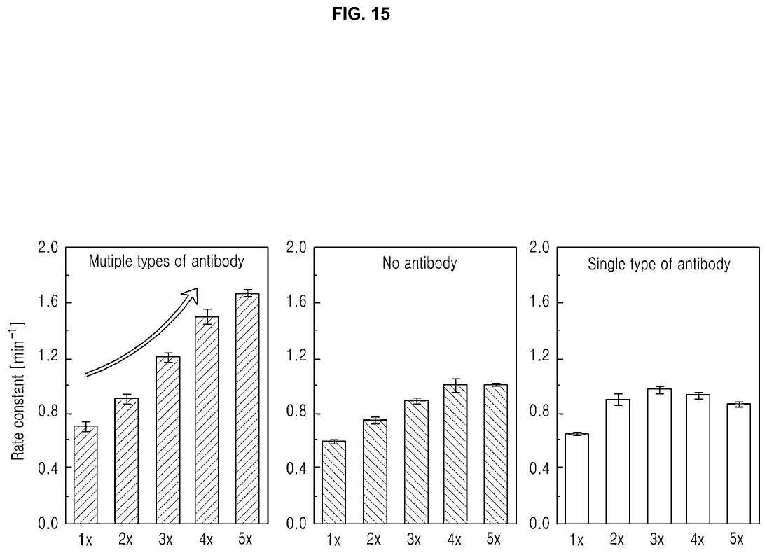

Furthermore, as illustrated in FIG. 15, the cells that metal particles were grown using several types of organelles by various types of antibodies show consistently improved catalytic activity. However, in the case of not using an antibody or using a single type of antibody, the number of seeds on which metal particles could be grown was limited or the space in which metal particles could be grown was limited. Accordingly, low catalyst activity was measured compared to the cells that metal particles were grown using various types of antibodies. Through such results, a method in which various antibodies can be used according to various embodiments can significantly increase catalyst performance because uniform metal particles are disposed in an independent space in which several biostructures are present.

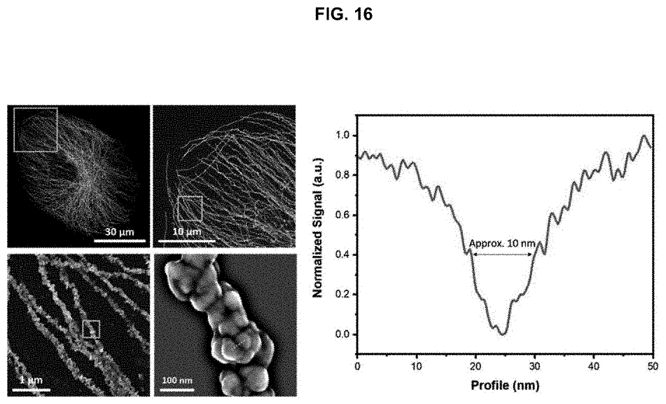

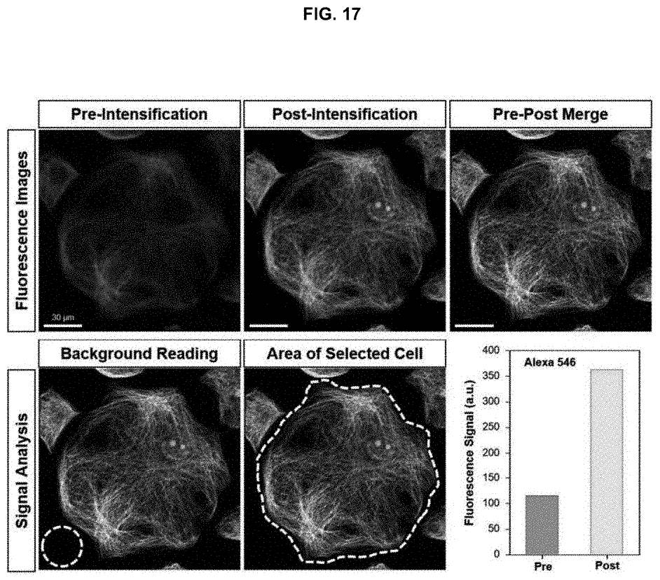

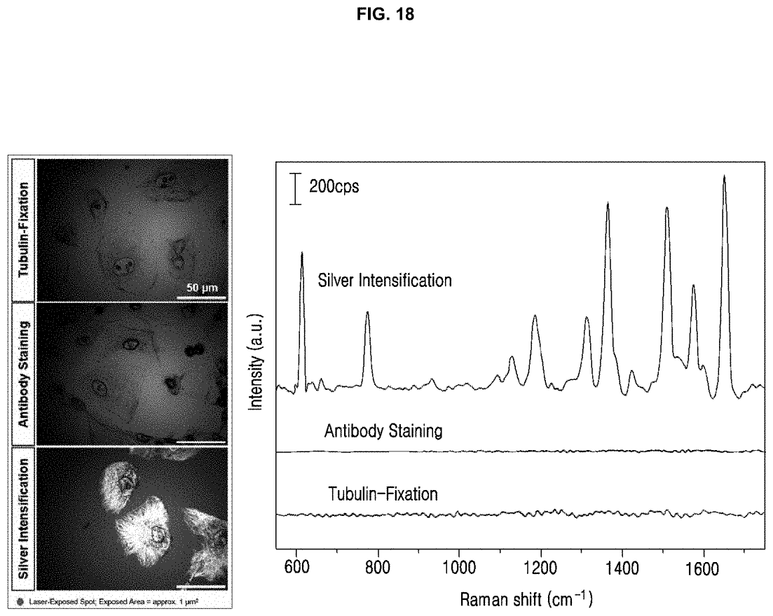

In the sixth experiment, an application as a surface-enhanced Raman scattering (SERS) substrate through a method according to various embodiments was confirmed. Through a method according to various embodiments, a biostructure was used as the SERS substrate by using characteristics of the biostructure. A microtubule structure within a cell has a form in which protein monomers called a tubulin are linearly self-assembled. Accordingly, a microtubule structure has a nanoscale repetitive interval. If metal particles are linearly present at such intervals, the biostructure can be efficiently used as an efficient SERS substrate because a large number of hot spots are included in the biostructure. First, as illustrated in FIG. 16, it was confirmed whether a distance between particles has an interval of a nano level in a structure synthesized along a microtubule through a method according to various embodiments. Furthermore, as illustrated in FIG. 17, the growth of silver particles having a higher effect was performed on Raman scattering than gold particles. It was confirmed that a biostructure could be efficiently used as a SERS substrate through signal amplification of a fluorescent material by using an antibody to which the fluorescent materials are together attached. As illustrated in FIG. 18, in order to check performance of the SERS substrate, rhodamine 6G used as a representative target. Characteristics was checked by measuring a Raman spectrum of the SERS substrate composed of cells on which only chemical fixation was performed, cells which microtubules were labeled with gold nano particles conjugated antibodies, and cells generated through the silver particle growth in the previous cells. The results showed that if only cells were used and if an antibody which gold nano particles were conjugated and labeled, a characteristic spectrum of rhodamine 6G could not be checked. In this case, it was found that if the interval between the particles was reduced through the silver particle growth in the gold nano particles, the biostructure could be used as the SERS substrate into which a structural characteristic of a microtubule is incorporated.

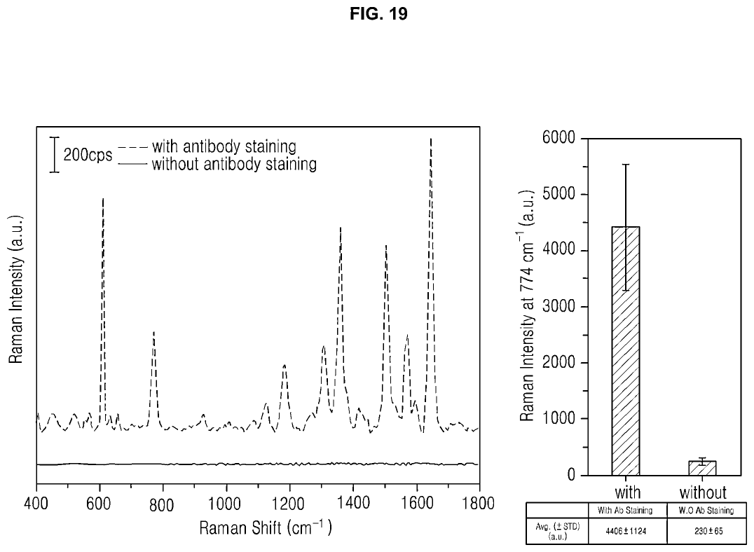

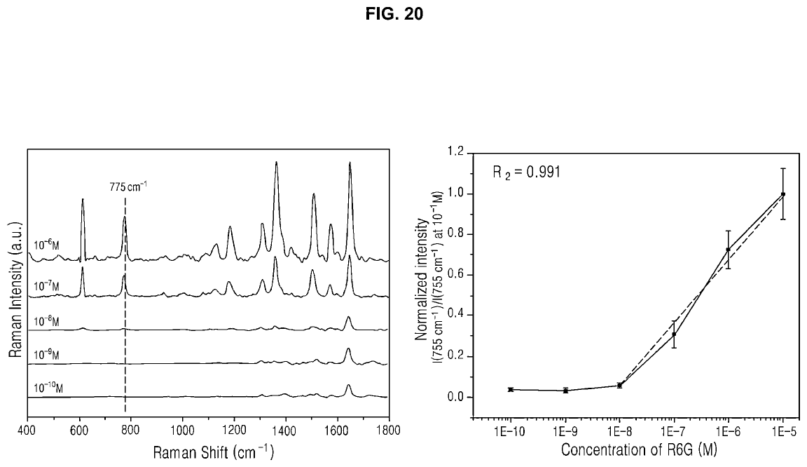

Furthermore, as illustrated in FIG. 19, through a comparison with a case where silver particle growth was performed on a cell on which only chemical fixation was performed without being labeled as an antibody which gold nano particles were conjugated as a control experiment, it was confirmed that silver particles were grown only in a microtubule structure with which gold nano particles was labeled. It is important to check whether linear detection for the SERS substrate is possible depending on a concentration of a detection material. As illustrated in FIG. 20, it was confirmed that a substrate generated through a method according to various embodiments could linearly detect through rhodamine 6G and a range thereof corresponding to a detection range of substrates generated through other technologies. If the method illustrated in the previous experiment is applied to the SERS substrate generated through a method according to various embodiments, it seems that a substrate having further excellent detection performance can be manufactured.

According to various embodiments, a structure having a functional material can be manufactured by using a complex structure in a biological specimen as a template in a way to label a biostructure within the biological specimen with an antibody and to grow an inorganic particle from the antibody. In this case, a specific characteristic can be further increased or a combination of several characteristics can be implemented with respect to the structure by selecting an inorganic particle. Furthermore, through immunostaining using an antibody pair, the structure can be manufactured in a form modified from the biostructure. Furthermore, the inorganic particle can be grown in various forms and can be synthesized as a multi-material particle by applying another material. That is, the structure can be manufactured from the biological specimen according to the desired application purpose. For example, a structure having a high mechanical characteristic and also high conductivity can be manufactured by synthesizing a structure having a metal material imitated from a spiral structure of collagen within a bone of an animal. For another example, an electronic circuit similar to a brain can be manufactured as a structure by synthesizing a complex structure of the metal, semiconductor, or non-conductive material, which has been imitated from a neuron within a brain.

Various embodiments provide a method of conversion and synthesis of materials via biostructures inside or outside a biological specimen. The method may include steps (steps 110, 210 and 220) of labeling an antibody which an inorganic particle has been conjugated in a biostructure, and steps (steps 130 and 230) of generating a structure corresponding to the biostructure by growing the inorganic particle with respect to the biostructure.

According to various embodiments, a biological specimen, that is, a target, may include prokaryotes such as viruses and bacteria, unicellular eukaryotes such as yeast, protoctists such as diatoms, insects, animal tissues, a plant, and human body tissues. The biological specimen may include cultured cells, cultured tissues, organoids, etc. which are artificially cultured on a plane or scaffold. Furthermore, the biological specimen may include 3D-printed cells or tissues, stacks of tissues, or an organ-on-a-chip obtained by culturing a cell or tissue on a chip. Also, the biological specimens may include such biological specimens on which gene-editing or processing has been performed. According to various embodiments, the step (steps 110, 210 and 220) of labeling the antibody may include labeling the antibody which an inorganic particle has been conjugated in a biostructure by using an antigen-antibody reaction.

According to various embodiments, the step (step 110) of labeling the antibody may include the biostructure by using a primary antibody which the inorganic particle has been conjugated without using a secondary antibody.

According to various embodiments, the primary or secondary antibody may include a common antibody, an antibody fragment, such as F(ab)₂, F(ab′)₂, Fab′, Fab, Fv, scFv, or VhH, and a minibody, a nanobody, affibody, diabody, and may include a nucleic acid-based aptamer.

According to various embodiments, the antibody may be substituted with any molecules capable of selectively labeling only a specific structure, for example, a nucleic acid sequence complementarily binding to a specific base sequence, a hydrophobic molecule selectively labeling lipids, etc.

According to various embodiments, instead of using inorganic particle-conjugated antibodies, a target molecule is expressed within cells through gene-editing and the target molecule is labeled with inorganic particle-conjugated another molecule that specifically binds to the target molecule.

According to various embodiments, the biostructure labeled as the antibody in the biological specimen may be composed of lipid, nucleic acid, an inorganic material, etc., including protein.

According to various embodiments, the step (steps 210 and 220) of labeling the antibody may include a step (step 210) of labeling a primary antibody in the biostructure, and a step (step 220) of staining the primary antibody by using a secondary antibody which an inorganic particle has been conjugated.

According to the first embodiment, the step (steps 210 and 220) of labeling the antibody may include a step (step 210) of labeling different biostructures by using different primary antibodies, and a step (step 220) of staining each of the primary antibodies by using a secondary antibody which an inorganic particle has been conjugated.

According to the second embodiment, the step (steps 210 and 220) of labeling the antibody may include a step (step 210) of labeling different biostructures by using different primary antibodies, and a step (step 220) of staining the primary antibody by using each of different secondary antibodies which different inorganic particles have been conjugated.

According to the third, fourth and fifth embodiments, the step (steps 210 and 220) of labeling the antibody may include a step (step 210) of labeling the biostructure by using the primary antibody, and a step (step 220) of staining the primary antibody by using a secondary antibody pair.

According to the third embodiment, the step (step 220) of staining the secondary antibody pair may include steps of staining the primary antibody by using a first secondary antibody which the inorganic particle has been conjugated, and staining the first secondary antibody by using a second secondary antibody which the inorganic particle has been conjugated.

According to the fourth embodiment, the step (step 220) of staining the secondary antibody pair may include steps of staining the primary antibody by using the first secondary antibody, and staining the first secondary antibody by using a second secondary antibody which the inorganic particle has been conjugated.

According to the third, fourth, and fifth embodiments, the step (steps 210 and 220) of labeling the antibody may further include a step (step 220) of additionally staining the secondary antibody pair by using a secondary antibody pair. The step (step 220) of additionally staining the secondary antibody pair may be repeated more than once.

According to the fifth embodiment, the step (step 220) of staining the secondary antibody pair may include steps of staining the primary antibody by using the first secondary antibody, and staining the first secondary antibody by using a second secondary antibody.

According to the fifth embodiment, the step 220 of additionally staining the secondary antibody pair may include a step of staining a previously stained second secondary antibody by using the first secondary antibody which the inorganic particle has been conjugated when the stain is the last stain.

According to the fifth embodiment, the step 120 of additionally staining the secondary antibody pair may include steps of staining a previously stained second secondary antibody by using the first secondary antibody, staining the first secondary antibody by using the second secondary antibody if additional staining is present, and staining the first secondary antibody by using the second secondary antibody which an inorganic particle has been conjugated when the stain is the last stain.

According to the third, fourth, and fifth embodiments, one type of antibody which the inorganic particle has been conjugated or two types of antibodies which two or more types of different inorganic particles have been conjugated, respectively, may be used in the first secondary antibody used in the step (steps 210 and 220) of labeling the antibody and the step (step 220) of additionally staining the secondary antibody pair by using the secondary antibody pair. Furthermore, one type of antibody which the inorganic particle has been conjugated or two types of antibodies which two or more types of different inorganic particles have been conjugated, respectively, may also be used in the second secondary antibody used in the step (steps 210 and 220) of labeling the antibody and the step (step 220) of additionally staining the secondary antibody pair by using the secondary antibody pair. Furthermore, the same inorganic particle may be conjugated or different inorganic particles may be conjugated to the first secondary antibody and the second secondary antibody used in the step (steps 210 and 220) of labeling the antibody and the step (step 220) of additionally staining the secondary antibody pair by using the secondary antibody pair.

According to various embodiments, the inorganic particle may include at least one of colloidal particles, nano particles, metal particles, non-conductive particles, semiconductor particles, quantum dots, or carbon particles.

According to the sixth embodiment, the step (steps 130 and 230) of generating the structure may include steps of growing an inorganic particle with respect to a biostructure and synthesizing a multi-material particle by applying another material to the grown inorganic particle. The structure may be generated from the multi-material particle.

According to the sixth embodiment, the step of synthesizing the multi-material particle may include a step of synthesizing the multi-material particle as a core-shell structure by applying another material to the outside of the grown inorganic particle.

According to the sixth embodiment, the step (step 230) of generating the structure may include a step of synthesizing the multi-material particle as an alloy structure by using different types of materials as the inorganic particle. The structure may be generated from the multi-material particle.

According to the seventh embodiment, the inorganic particle may be grown to have, as the inorganic particle, at least one form of a spherical shape, a rod shape, a sheet shape, or a pyramid shape.

For example, the inorganic particle may be a gold nano particle.

A method according to various embodiments is related to various industry fields.

The first is an antibody industry. A method of synthesizing an inorganic material based on a biostructure by using an antibody is used in a method according to various embodiments. If a characteristic of a biostructure is used, it is essential to develop an antibody that clearly labels protein forming the biostructure. Furthermore, although a new biostructure is found and shows a structural characteristic, an antibody is necessary when a structure is generated using another material. Due to several factors including the development of a bioengineering technology, such as proteomics and genomics which may be associated with a method according to various embodiments, a global antibody and reagent market volume for research is predicted to reach 14.193 billion dollars in 2025. The development of the antibody industry is expected to play a positive role that expands the accuracy or variety of a method according to various embodiments. For example, if a monoclonal antibody production technology is developed, protein to be targeted within a biostructure can be more clearly synthesized as another material because the selectivity of the protein. Furthermore, if the size of an antibody can be made smaller than that of the existing antibody like a nanobody antibody, a biostructure to be imitated through a method according to various embodiments can be more finely synthesized as a structure having another material. Finally, if an antibody stably combined with various inorganic materials, organic particles, etc. is developed, a structure can be synthesized as particles having various functionalities based on a biostructure through a method according to various embodiments.

The second is a genetic engineering-related industry. Genetic engineering corresponding to a representative technology that will lead the 4^{th} industrial revolution is also associated with the present disclosure, and may representatively include transformation and gene editing technologies. The transformation technology may be used as a method of expressing protein capable of being labeled in a corresponding structure if an antibody with which the desired structure will be labeled is not present. Furthermore, the transformation technology may be used to improve the precision of structure synthesis according to various embodiments according to a transgenic method of generating protein capable of recognizing a single chemical functional group attached to an inorganic material like SNAP-tag or CLIP-tag out of a structure synthesis method using an antibody. After a specific structure is removed within a biological sample, a specific structure is changed into a desired shape or a specific structure is added by using technologies, such as CRISPR that is a gene editing technology, the present disclosure may be applied and used to improve the selectivity of the present disclosure.

The third is a tissue engineering-related industry. Like genetic engineering, a tissue engineering-related industry corresponding to the category of bioengineering technology is also related to the present disclosure. Tissue engineering also includes technologies, such as three-dimensional cell culture and biological specimen printing. It is expected that such bio tissue market will exceed 4.8 billion dollars until the year of 2028. This positively influences an increase of the variety and universality of a biostructure which may be used in various embodiments. For example, in various embodiments, when it is difficult to find the desired structure in a biostructure present in the natural world, a biostructure may be artificially generated through tissue engineering technology and an inorganic material structure may be manufactured. Furthermore, through a method according to various embodiments, a uniform form is obtained. If mass production is required, an alternative using a tissue artificially generated through tissue engineering may be selected.

A method according to various embodiments may be applied to various industry fields.

The first is the catalyst industry. As described above, it is expected that biostructures grown from several inorganic particles based on a biostructure by applying a method according to various embodiments as in two liquid-phase reactions may be used in various catalyst reactions. In particular, a method of synthesizing, within a living body, particles which are well distributed by simultaneously growing inorganic particles in biostructures present in independent spaces and which have a size of a nano level according to various embodiments cannot be synthesized using the existing particle growth technology or bio-template technology. Furthermore, if a method of growing different inorganic particles in each type of protein present in various embodiments is used, it is expected that the different inorganic particles can be synthesized as a multi-functional catalyst on which various catalyst reactions can be performed. Furthermore, if a method according to various embodiments is applied to a biomaterial such as a tissue having a large dimension in a cell level, it is expected that particles can be grown based on more various and many structures. Accordingly, the variety and activity of a catalyst reaction will be increased.

The second is a SERS substrate industry. As described above, if a biostructure capable of densely generating hot spots in addition to a microtubule structure is used through a method according to various embodiments, an efficient SERS substrate into which a structural characteristic has been incorporated can be manufactured. Furthermore, after a cell is cultured in a patterned substrate and a method according to various embodiments is applied to the cell, uniform detection performance in the entire substrate can be expected.

The third is the battery industry. There are many bio-templating methods for synthesizing a biostructure having a fiber form in a net form and using it in an electrode of a battery. Most representatively, there is research in which after gene editing using M13 virus having a long cylindrical structure, a battery is manufactured by synthesizing various inorganic materials on the outer wall of the virus. However, it seems that the research has not been commercialized due to the following three limits. 1) There was a limit in mass production as an industry volume apparatus. 2) Gene editing processing was necessary in order to perform inorganic material synthesis in a virus. 3) An aggregation phenomenon between viruses was severe. Various embodiments can solve the above three limits. First, a large-scale biological specimen can be used, such as a tissue which can be easily used. Second, gene editing is not required before inorganic material synthesis if a commercialized antibody is present because the targeting of a specific structure is performed using the antibody. Third, since structures within a cell have already been independently organized and present, an aggregation phenomenon between the structures can be suppressed. If a biostructure having a fiber form, such as a microtubule, is used based on such advantages, the present disclosure may be used as a battery electrode like virus research and can also be commercialized.

A structure manufactured by a method according to various embodiments can be variously used.

First, the structure may be used as an eco-friendly catalyst scaffold. In order to use nano particles in a catalyst, it is essential to reduce an aggregation phenomenon between particles. In order to achieve the reduction, nano particles are strongly combined with a scaffold and used as a catalyst. However, a condition in which the synthesis of catalyst substrate is complicated, and a material having toxicity is chiefly used. If a method according to various embodiments is used, biomaterials which may be easily obtained nearby may be used as the catalyst scaffold capable of preventing the aggregation of nano particles.

Second, the structure may be used as a SERS substrate capable of selective detection. In the present disclosure in which an antibody is used, a method of combining DNA a lot used in research with an antibody in order to selectively detect a material for a SERS substrate. If metal particles and an antibody combined with DNA are simultaneously introduced, detection selectivity can be increased. After DNA is designed as a sequence listing that detects different molecules, if the DNA is combined with antibodies that label different types of protein, a substrate that simultaneously detects several materials may be manufactured.

Third, the structure may be used as a battery electrode having a high porosity. First, it seems that a battery electrode close to commercialization can be produced by supplementing a problem of the existing bioframe through a method according to various embodiments. Furthermore, as a battery having higher output and capacity is required, if a biostructure that is dense or has high porosity so that ions can be easily moved is used as a frame, it is considered that the structure may be developed as a competitive battery material.

As described above, a method according to various embodiments presents a new bio-templating method. A method according to various embodiments presents a new method for a bio-templating method that is being stalled as imitating only an external structure, and has a new method having a high value, which can achieve a biomimetic technology and various academic harmonies. Furthermore, a method according to various embodiments presents a wide application scheme through the coupling of structural characteristics of biostructures and functional characteristics of particles. A method according to various embodiments may be used in extensive application field through biostructures present in the natural world, structural characteristics of artificial biostructures generated by various bioengineering technologies, and various particle growth strategies, in addition to the aforementioned application to the catalyst, the battery, and the SERS substrate.

Various embodiments of this document and the terms used in the embodiments are not intended to limit the technology described in this document to a specific embodiment, but should be construed as including various changes, equivalents and/or alternatives of a corresponding embodiment. Regarding the description of the drawings, similar reference numerals may be used in similar elements. An expression of the singular number may include an expression of the plural number unless clearly defined otherwise in the context. In this document, an expression, such as “A or B”, “at least one of A and/or B”, “A, B or C” or “at least one of A, B and/or C”, may include all of possible combinations of listed items together. Expressions, such as “a first,” “a second,” “the first” or “the second”, may modify corresponding elements regardless of its sequence or importance, and are used to only distinguish one element from the other element and do not limit corresponding elements. When it is described that one (e.g., a first) element is “(functionally or communicatively) connected to” or “conjugated with” the other (e.g., a second) element, one element may be directly connected to the other element or may be connected to the other element through another element (e.g., a third element).

According to various embodiments, each of the aforementioned elements may include a single entity or a plurality of entities. According to various embodiments, one or more of the aforementioned components or steps may be omitted or one or more other components or steps may be added. Alternatively, or additionally, a plurality of components may be integrated into a single component. In such a case, the integrated component may identically or similarly perform a function performed by a corresponding one of the plurality of components before one or more functions of each of the plurality of components. According to various embodiments, steps performed by a module, a program or another component may be executed sequentially, in parallel, iteratively or heuristically, or one or more of the steps may be executed in different order or may be omitted, or one or more other steps may be added.

The embodiments of the disclosure in which an exclusive property or privilege is claimed are defined as follows:

1. A method of conversion and synthesis of materials via biostructures, comprising:
labeling a biostructure by using an antibody which an inorganic particle has been conjugated; and
generating a structure corresponding to the biostructure by growing the inorganic particle with respect to the biostructure.

2. The method of claim 1, wherein the labeling of the biostructure by using the antibody comprises labeling the biostructure by using the antibody which the inorganic particle has been conjugated based on an antigen-antibody reaction.

3. The method of claim 1, wherein the labeling of the biostructure by using the antibody comprises:
labeling the biostructures by using a primary antibody; and
staining the primary antibody by using a secondary antibody which the inorganic particle has been conjugated.

4. The method of claim 1, wherein the labeling of the biostructure by using the antibody comprises:
labeling different biostructures by using different primary antibodies; and
staining each of the primary antibodies by using a secondary antibody which the inorganic particle has been conjugated.

5. The method of claim 1, wherein the labeling of the biostructure by using the antibody comprises:
labeling different biostructures by using different primary antibodies; and
respectively staining the primary antibodies by using different secondary antibodies which different inorganic particles are conjugated.

6. The method of claim 1, wherein the labeling of the biostructure by using the antibody comprises:
labeling the biostructure by using a primary antibody; and
staining the primary antibody by using a secondary antibody pair.

7. The method of claim 6, wherein the staining of the primary antibody by using the secondary antibody pair comprises:
staining the primary antibody by using a first secondary antibody; and
staining the first secondary antibody by using a second secondary antibody which an inorganic particle has been conjugated.

8. The method of claim 6, wherein the staining of the primary antibody by using the secondary antibody pair comprises:
staining the primary antibody by using a first secondary antibody which an inorganic particle has been conjugated; and
staining the first secondary antibody by using a second secondary antibody which an inorganic particle has been conjugated,
wherein the inorganic particle conjugated with the first secondary antibody and the inorganic particle conjugated with the second secondary antibody are identical with or different from each other.

9. The method of claim 6, wherein the labeling of the biostructure by using the antibody further comprises additionally staining the secondary antibody pair by using a secondary antibody pair, and
wherein the additionally staining of the secondary antibody pair is repeated more than once.

10. The method of claim 9, wherein an inorganic particle has been conjugated with the secondary antibody pair, and
wherein the inorganic particle conjugated with the additionally stained secondary antibody pair and the inorganic particle conjugated with the previously stained secondary antibody pair are identical with or different from each other.

11. The method of claim 9, wherein the staining of the primary antibody by using the secondary antibody pair comprises:
staining the primary antibody by using a first secondary antibody; and
staining the first secondary antibody by using a second secondary antibody,
wherein the additionally of staining the secondary antibody pair comprises staining the second secondary antibody by using a first secondary antibody which an inorganic particle has been conjugated when the additional staining is a last staining.

12. The method of claim 9, wherein the staining of the primary antibody by using the first secondary antibody comprises:
staining the primary antibody by using a first secondary antibody; and
staining the first secondary antibody by using a second secondary antibody,
wherein the additionally staining of the secondary antibody pair comprises:
staining the second secondary antibody by using a first secondary antibody;
staining the first secondary antibody attached to the second secondary antibody by using a second secondary antibody when an additional staining is present; and
staining the first secondary antibody attached to the second secondary antibody by using a second secondary antibody which an inorganic particle has been conjugated when the staining is a last staining.

13. The method of claim 1, wherein the inorganic particle comprises at least one of a colloidal particle, a nano particle, a metal particle, a non-conductive particle, a semiconductor particle, a quantum dot, or a carbon particle.

14. The method of claim 1, wherein the generating of the structure comprises:
growing the inorganic particle with respect to the biostructures; and
synthesizing a multi-material particle by applying another material to the grown inorganic particle,
wherein the structure is generated from the multi-material particle.

15. The method of claim 12, wherein the synthesizing of the multi-material particle comprises synthesizing the multi-material particle as a core-shell structure by applying another material to an outside of the grown inorganic particle.

16. The method of claim 1, wherein generating the structure comprises synthesizing the multi-material particle as an alloy structure by using different materials as the inorganic particle, and
wherein the structure is generated from the multi-material particle.

17. The method of claim 1, wherein the inorganic particle is grown to have at least one form of a spherical shape, a rod shape, a sheet shape, or a pyramid shape.

18. The method of claim 1, wherein the inorganic particle is a gold nano particle.

19. The method of claim 1, wherein the biostructure is composed of at least one of protein, lipid, nucleic acid, or an inorganic material.

20. A structure manufactured according to the method of claim 1.
